(19) United States
(12) Patent Application Publication
(10) Pub. No.: US 2006/0100523 A1
(43) Pub. Date: May 11, 2006

(54) Noninvasive blood vessel location device and method

(76) Inventors: John Seldon Ogle, Milpitas, CA (US); Martin Ademovic, Pittsford, NY (US)

Correspondence Address: John Seldon Ogle, 1472 Pashote Court, Milpitas, CA, 96035, US

(21) Appl. No.: 10/985,222

(22) Filed: Nov. 8, 2004

(51) Int. Cl. A61B 6/00

(52) U.S. Cl. 600/473; 600/476

(57) ABSTRACT

A handheld device for sensing and indicating the location of a blood vessel, and marking the skin adjacent to the blood vessel. The operation of the device is based on determining the difference in signal transfer amplitude between two partially overlapping infrared light paths, and moving the device until this difference is zeroed. The sensitivity of the device is enhanced by having an angle in each infrared light path such that the infrared light is primarily reflected, rather than absorbed, by the blood vessel.

### CROSS-REFERENCE TO RELATED APPLICATIONS

Not Applicable

### FEDERALLY SPONSORED RESEARCH

Not Applicable

### SEQUENCE LISTING OR PROGRAM

Not Applicable

### BACKGROUND OF THE INVENTION—FIELD OF THE INVENTION

This invention relates to the noninvasive sensing of blood vessel location, primarily to facilitate the accurate insertion of a hypodermic needle into the blood vessel.

### BACKGROUND OF THE INVENTION—PRIOR ART

A common medical testing procedure involves drawing blood to determine the constituents in the blood. In addition to the normal constituents of blood, it may be desirable to determine the presence in the blood of bacteria, viruses, alcohol, or various drugs.

For certain individuals, especially for infants and for those with thick fat layers, it is difficult to accurately locate a blood vessel for insertion of a hypodermic needle. This is commonly a problem for inexperienced medical personnel.

A number of procedures and apparatus types have been developed to facilitate the accurate location of blood vessels, the most successful of which use infrared light to noninvasively sense blood vessel locations. These infrared blood vessel sensors operate on the principle that, over a range of infrared wavelengths from about 700 nanometers to about 1200 nanometers, both the skin surface and flesh between the skin surface and a blood vessel are translucent, while the blood in a blood vessel absorbs the infrared light. This principle has been demonstrated through the use of infrared night vision scopes, which can clearly show a pattern of subsurface blood vessels.

An example of this approach is described in U.S. Pat. No. 6,230,046 to Crane et al (2001). The equipment required is expensive, cumbersome, and delicate, and does not provide an easy means to mark the blood vessel location.

Even more complicated and cumbersome equipment is described in U.S. Pat. 6,424,858 to Williams (2002), U.S. Pat. No. 6,463,309 to Ilia (2002) and U.S. Pat. No. 6,522,911 to Toida et al (2003). All of these systems suffer from a low contrast between the blood vessel and the surrounding flesh because they rely on the relative absorption and scattering of infrared light between the blood and the surrounding flesh.

Transillumination, with the infrared light source and the photosensitive element on opposite sides of the body part containing the blood vessel, can provide improved contrast between the blood vessel and the surrounding flesh, but both the light intensity and the contrast vary with the thickness of the body part, with additional noise introduced by irregularities such as bones.

For reflective illumination, with the infrared source and the photosensitive element on the same side of the body part, the contrast relies on infrared light scattering beyond the blood vessel to return enough light to provide a detectable shadow from the blood vessel. U.S. Pat. No. 5,519,208 to Esparza et al (1996) describes how the blood vessels show up as dark lines, and suggests the use of an image intensifier to provide usable contrast.

Each of the prior art approaches is expensive and inconvenient to use, while providing marginal contrast between blood vessels and surrounding flesh.

### BACKGROUND OF THE INVENTION—OBJECTS AND ADVANTAGES

Several objects and advantages of the invention are:
(a) to provide a clear signal indicating the location of a blood vessel;
(b) to provide a small, convenient blood vessel location sensor, with a clear indication, usable with little training; and
(c) to provide a convenient means for marking the skin above the blood vessel to guide the insertion of a hypodermic needle;

### SUMMARY

In accordance with the present invention, a handheld blood vessel location sensor is provided with a visual indication of lateral displacement from above the blood vessel. Two laterally spaced infrared sensing paths are provided in the blood vessel location sensor and the difference in transfer functions between these infrared sensing paths drives the visual indication of lateral displacement.

### DRAWINGS

FIG. 1 is a perspective drawing of one embodiment of the blood vessel location sensor.

FIG. 2 is an elevation view showing the relative angles and positions of the optical elements of infrared sense path 20.

FIG. 3 is an elevation view showing the relative locations of the blood vessel and a typical sensitivity pattern for each infrared sensing path.

FIG. 4 is a block diagram of typical electrical connections for the sensor.

FIG. 5 is a timing diagram showing typical LED pulse drive and phototransistor output measurement times.

### DRAWINGS—Reference Numerals

10 blood vessel location sensor
12 blood vessel location display LED array
14 blood vessel location marker button
16 infrared LED
18 infrared phototransistor
20 infrared sense path
22 blood vessel
24 included angle
26 microprocessor
28 LED drivers
30 infrared LED drive pulse
32 infrared phototransistor zero sense interval
34 infrared phototransistor sense interval
36 skin surface

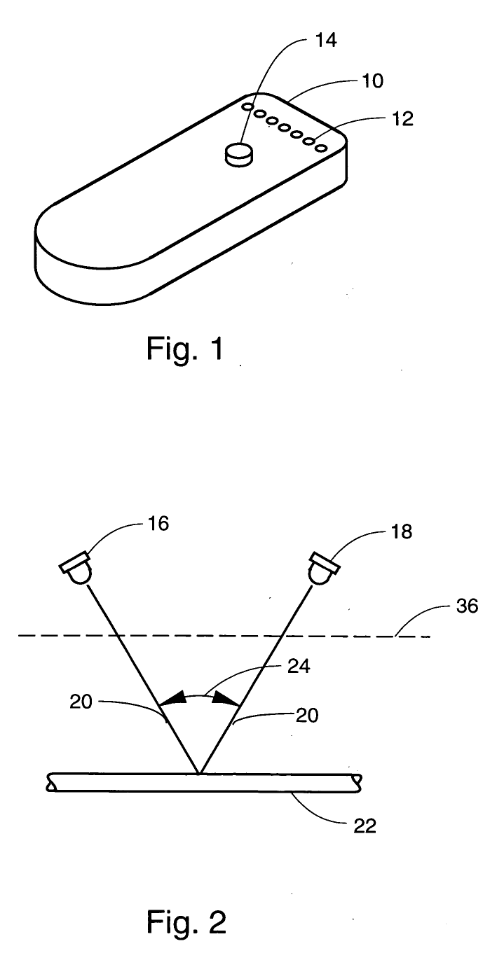

### DETAILED DESCRIPTION—FIGS. 1 through 5—PREFERRED EMBODIMENT

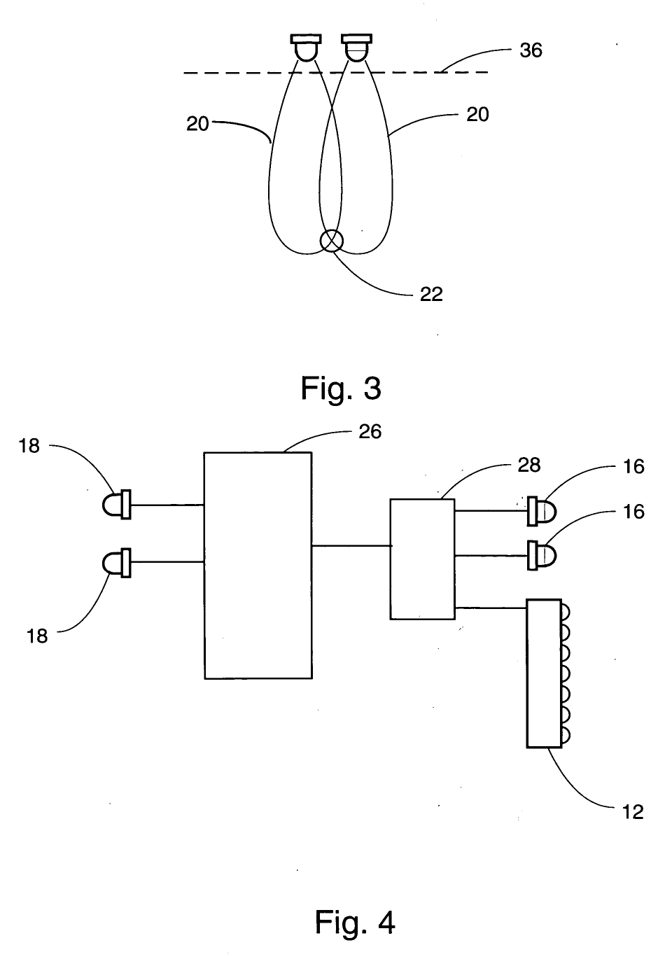

FIG. 1 is a perspective view of a preferred embodiment of blood vessel location sensor 10. Blood vessel location display LED array 12 provides a visual indication of lateral displacement between the blood vessel and the lateral center of blood vessel location sensor 10. Blood vessel location marker button 14 provides a means for marking the skin at a point beneath the lateral center of blood vessel location sensor 10. Marker button 14 can activate a felt tip pen, a pressure sensitive ink strip, or similar means to mark the skin. FIG. 2 is an elevation view of infrared sense path 20 with a 70 degree included angle 24 between the axis of infrared LED 16 and the axis of infrared phototransistor 18, with blood vessel 22 reflecting the infrared light. Infrared LED SFH 409 is suitable for infrared LED 16, and matching phototransistor SFH 309 is suitable as infrared phototransistor 18. These components are available from Osram Opto Semiconductors Gmbh, Wemerwerkstrasse 2, D-93049 Regensburg, Germany. The SFH 409 emits at a narrow wavelength centered at 950 nanometers, with a half angle of 20 degrees. FIG. 3 is an elevation view showing the relative location between each infrared sense path 20 and blood vessel 22. FIG. 4 is a block diagram showing the electrical connections between microprocessor 26, LED drivers 28, each infrared LED 16, each infrared phototransistor 18, and blood vessel location display LED array 12. Microprocessor 26 incorporates analog to digital converters which convert signals from each infrared phototransistor 18 to digital form.

A Freescale (Motorola) DSP56F801 is suitable for use as microprocessor 26.

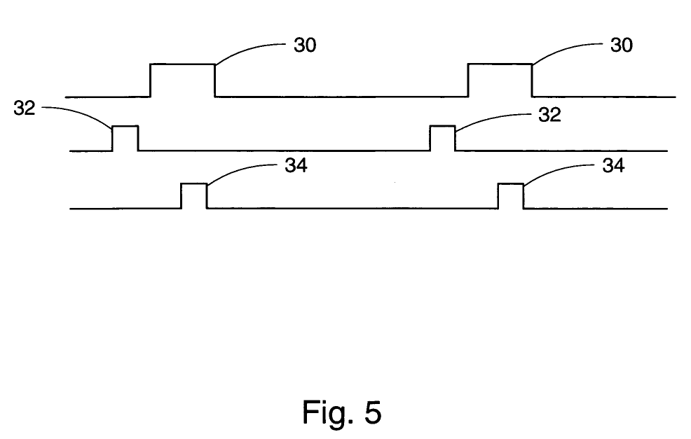

FIG. 5 is a timing diagram showing how the effects of ambient infrared light are cancelled. During infrared phototransistor zero sense interval 32, with infrared drive pulse 30 at zero, a zero, or passive, signal from each infrared phototransistor 18 is measured. Then infrared LED drive pulse 30 is applied to each infrared LED 16, and an active output of each infrared phototransistor 18 is measured, during infrared phototransistor sense interval 34. The differences between active and passive measurements for each infrared phototransistor 18 are calculated as effective signal amplitudes. The difference between effective signal amplitudes for the two units of infrared phototransistor 18 are used to drive blood vessel location display LED array 12.

For a small included angle 24 between the axis of infrared LED 16 and the axis of infrared phototransistor 18, the blood in blood vessel 22 absorbs the infrared light, and reflects less infrared light than the adjacent flesh, in which the light is scattered. Thus the blood vessel appears dark relative to a scattered light background. However, at an included angle 24 of around 70 degrees, the infrared light from infrared LED 16 is reflected from blood vessel 22, rather than being absorbed, resulting in greater infrared light from blood vessel 22 to infrared phototransistor 18 than for the surrounding flesh. Thus the blood vessel appears bright rather than dark. This results in greatly increased sensitivity of reflected infrared light to blood vessel 22 location.

To assure that a minimum error signal means that blood vessel location sensor 10 is positioned over blood vessel 22, rather than that there is no infrared light reflected from blood vessel 22, the output of each infrared phototransistor 18 is summed, the sum is compared against a reference value, and an indicator is activated to indicate satisfactory reflected light signal strength.

### OPERATION

Blood vessel location sensor 10 is moved until blood vessel location display LED array 12 indicates no lateral displacement relative to blood vessel 22. Blood vessel location marker button 14 is then pressed to mark skin surface 36 over blood vessel 22. This mark is then used as a reference point for hypodermic needle insertion.

FIGS. 1,3—Additional Embodiments

Blood vessel location display LED array 12 can use display LEDs of various colors to enhance display information.

The function of blood vessel location display LED array 12 can also be provided by other display technologies such as an analog meter, a liquid crystal display, or an acoustical indication of lateral displacement of blood vessel location sensor 10 relative to blood vessel 22.

I claim:

1. A blood vessel location sensor incorporating two infrared sensing paths, each of said infrared sensing paths including an infrared light source with a directional light pattern and an infrared light sensor with a directional sensitivity, with an included angle between the axis of said infrared light source and the axis of said infrared light sensor of between 30 degrees and 130 degrees,
said two infrared sensing paths spaced apart to provide a partial sensing overlap at the expected blood vessel distance below the skin,
means for measuring and displaying the difference in signal amplitudes between the outputs of the infrared light sensors of said two infrared sensing paths.

2. The blood vessel location sensor of claim 1, with the addition of means for marking the skin to indicate the location of said blood vessel location sensor relative to the skin.

3. The blood vessel location sensor of claim 1, in which said infrared light source is a light emitting diode.

4. The blood vessel location sensor of claim 1, in which said infrared light sensor is a phototransistor.

5. The blood vessel location sensor of claim 1, with the addition of means for driving said infrared light source with modulated power, with the output signal from said infrared light sensor measured both with drive to said infrared light source and without drive to said infrared light source, and effective signal amplitudes from said infrared light sensors equal to the difference between said signal amplitude measurement at the time said drive is applied to said infrared light source and the time said drive is not applied to said infrared light source.

6. The blood vessel location sensor of claim 1, with the addition of means for measuring the sum of the output signals of each said infrared light sensor and providing an indication if this sum is greater than a reference value.
